(19) United States
(12) Patent Application Publication
(10) Pub. No.: US 2013/0273607 A1
(43) Pub. Date: Oct. 17, 2013

(54) METHODS FOR PROCESSING INCLUSION BODIES

(71) Applicant: Ellen T. O'Connor, Gaithersburg, MD (US)

(72) Inventor: Ellen T. O'Connor, Gaithersburg, MD (US)

(73) Assignee: MEDIMMUNE, LLC, Gaithersburg, MD (US)

(21) Appl. No.: 13/880,424

(22) PCT Filed: Oct. 20, 2011
(86) PCT No.: PCT/US11/57004

§ 371 Date: Jul. 3, 2013

Related U.S. Application Data
(60) Provisional application No. 61/394,878, filed on Oct. 20, 2010.

(51) Int. Cl. C07K 1/36

(52) U.S. Cl. CPC C07K 1/36
USPC 435/69.6; 530/389.6; 252/182.12

(57) ABSTRACT

The present application relates to methods for purifying recombinant proteins, including antibodies and antibody fragments. Suitably, the methods utilize depth filtration to clarify the desired proteins from a solubilized mixture, and provide refolding methods and refolding buffers to allow for refolding of the recombinant proteins into functional and active proteins. Exemplary antibody fragments include anti-CD22 antibody fragments that comprise V_{H} and V_{L} chains refolded into a functional and active fragment.

### BACKGROUND OF THE INVENTION

1. Field of the Invention

The present application relates to methods for purifying recombinant proteins, including antibodies and antibody fragments. Suitably, the methods utilize depth filtration to clarify the desired proteins from a solublized mixture, and provide refolding methods and refolding buffers to allow for refolding of the recombinant proteins into functional and active proteins.

2. Background of the Invention

Purification of recombinant proteins, and particularly antibodies and antibody fragments, can be a highly involved, labor-intensive and time-consuming process. Production of recombinant proteins from bacterial cell cultures results in the desired proteins being associated with inclusion bodies from the cell. Generally, separation of the desired proteins from the inclusion body utilizes clarification with a combination of centrifugation and depth filtration, depth filtration and tangential flow filtration, or tangential flow filtration alone. Such combination methods for clarification are typically utilized so as to enable sufficient clarification and prevent fouling of purification columns further downstream. Following the separation, the proteins can then be refolded into an active and functional form. For clarification of complex proteins, for example antibody fragments comprising variable heavy (V_{H}) and variable light (V_{L}) chains, minimizing the number of purification steps generally results in increased product yield and purity, and reduced production times and costs.

U.S. Pat. No. 7,355,012 (hereinafter “the '012 patent”), the disclosure of which is incorporated by reference herein in its entirety, discloses antibodies for binding to CD22-expressing cells, especially cancer cells that express CD22 on their exterior surface. The '012 patent describes anti-CD22 antibodies with a V_{L} chain having the sequence of antibody RFB4 and a VH chain having the sequence of antibody RFB4, wherein residues 100, 100A and 100B of CDR3 of the V_{H} chain (as numbered by the Kabat and Wu numbering system) have an amino acid sequence selected from the group consisting of THW, YNW, TTW, and STY. The '012 patent describes production of the anti-CD22 antibodies via generation of the entire, full-length antibody fragment via recombinant methods, followed by purification and refolding. However, for preparation of recombinant proteins in which portions (for example V_{H} and V_{L} portions of an antibody or antibody fragment) are separately produced in different cell cultures (or even in the same cell culture), purification methods are needed to provide efficient, high purity and high yield protein production.

### SUMMARY OF PREFERRED EMBODIMENTS

The needs identified above by met by the present application providing methods of purification of recombinant proteins.

In embodiments, methods are provided for purifying a recombinant protein from a mixture comprising the recombinant protein and inclusion bodies. The methods suitably comprise solubilizing the mixture comprising the recombinant protein with associated inclusion bodies with a solubilization buffer, clarifying the recombinant protein from the solubilized mixture with one or more depth filters and recovering the clarified recombinant protein. Suitably, the methods do not include centrifuging the solubilized mixture of recombinant protein with associated inclusion bodies prior to the clarifying.

In exemplary embodiments, the recombinant protein comprises an antibody or an antibody fragment, including a heavy chain (V_{H}) antibody fragment or a light chain (V_{L}) antibody fragment, such as, for example, anti-CD22 antibody fragments.

Exemplary solublization buffers comprise ethanolamine, arginine, EDTA, urea and DIE, for example, about 20 mM to about 70 mM ethanolamine, about 200 mM to about 2 M arginine, about 1 mM to about 3mM EDTA, about 5 M to about 10 M urea and about 5 mM to about 20 mM DTE, at a pH of about 10 to about 11.

In embodiments, the recombinant protein is clarified with two or more depth filters, suitably in series, such as a first depth filter comprising cellulose fiber and diatomaceous earth, and having a nominal micron rating of about 0.1 μm to about 1 μm (e.g., a C0HC depth filter (MILLIPORE®)) and a suitable second depth filter comprises cellulose fiber and diatomaceous earth, and has a nominal micron rating of less than about 0.1 μm (e.g., an X0HC depth filter (MILLIPORE®)).

The methods suitably further comprise diluting a concentrated, clarified recombinant protein in a protein refolding buffer comprising about 20 mM to about 70 mM ethanolamine, about 0.5 M to about 2 M arginine, about 0.5 mM to about 3 mM EDTA, and about 0.5 mM to about 1.5 mM GSSG, incubating the diluted clarified recombinant protein at a pH of about 9 to about 10 and at a temperature of about 2° C. to about 15° C., for about 48 hours to about 96 hours, and recovering the recombinant protein.

Also provided are methods of producing a recombinant antibody fragment comprising a V_{H} antibody fragment and a V_{I} antibody fragment. The methods suitably comprise expressing a polynucleotide encoding a V_{H} antibody fragment in a first bacterial cell and expressing a polynucleotide encoding a V_{L} antibody fragment in a second bacterial cell. The V_{H} antibody fragment and the V_{L} antibody fragment are mixed to generate a mixture, wherein the mixture further comprises inclusion bodies. The mixture comprising the VH antibody fragment, the V_{L} antibody fragment with associated inclusion bodies is solublized with a solubilization buffer. The V_{H} antibody fragment and the V_{L} antibody fragment are clarified from the solubilized mixture with one or more depth filters, and the clarified V_{H} antibody fragment and the clarified V_{L} antibody fragment are recovered. Suitably, the method does not include centrifuging the solubilized mixture of V_{H} antibody fragment, V_{L} antibody fragment and inclusion bodies prior to clarification using depth filtration.

The clarified V_{H} antibody fragment and the clarified V_{L} antibody fragment are concentrated, and then diluted with a refolding buffer comprising: about 20 mM to about 70 mM ethanolamine; about 0.5 M to about 2 M arginine; about 0.5 mM to about 3 mM EDTA; and about 500 mM to about 1.5 mM GSSG. The diluted clarified V_{H} antibody fragment and the diluted clarified V_{L} antibody fragment are incubated at a pH of about 9 to about 10 and at a temperature of about 10° C. to about 15° C., for about 48 hours to about 96 hours. The recombinant antibody fragment is then recovered.

As described herein, suitably the V_{H} antibody fragment is an anti-CD22 V_{H} antibody fragment and the V_{L} antibody fragment is an anti-CD22 V_{L} fragment, and the recombinant antibody fragment is an anti-CD22 antibody fragment. Exemplary solublization and refolding buffers are described herein, as are methods for depth filtration.

Further embodiments, features, and advantages of the embodiments, as well as the structure and operation of the various embodiments, are described in detail below with reference to accompanying drawings.

### BRIEF DESCRIPTION OF THE FIGURES

FIG. 1A shows the results of reverse phase high-performance-liquid-chromatography mass spectroscopy (RP-HPLC-MS) analysis of anti-CD22 antibody following refolding under a first set of refolding conditions.

FIG. 1B shows the results of RP-HPLC-MS analysis of anti-CD22 antibody following refolding under a second set of refolding conditions.

FIG. 1C shows the results of a RP-HPLC-MS analysis of anti-CD22 antibody following refolding under a third set of refolding conditions.

FIG. 1D shows the results of a RP-HPLC-MS analysis of anti-CD22 antibody following refolding under a fourth set of refolding conditions.

FIG. 2A shows the results of a RP-HPLC-MS analysis of anti-CD22 antibody following refolding at time=0.

FIG. 2B shows the results of a RP-HPLC-MS analysis of anti-CD22 antibody following refolding at time=20 hours.

FIG. 2C shows the results of a RP-HPLC-MS analysis of anti-CD22 antibody following refolding at time=90 hours.

FIG. 3A shows the results of RP-HPLC-MS indicating the presence of an anti-CD22 antibody fragment after further purification.

FIG. 3B shows the results of RP-HPLC-MS indicating the presence of an anti-CD22 antibody fragment after further purification.

FIG. 4A shows RP-HPLC-MS analyses of a 5 kg refold reaction at time=0.

FIG. 4B shows RP-HPLC-MS analyses of a 5 kg refold reaction at time=15 hours.

FIG. 4C shows RP-HPLC-MS analyses of a 5 kg refold reaction at time=24 hours.

FIG. 4D shows RP-HPLC-MS analyses of a 5 kg refold reaction at time=39 hours.

FIG. 5A shows RP-HPLC-MS analyses of a 100 kg refold reaction at time=0.

FIG. 5B shows RP-HPLC-MS analyses of a 100 kg refold reaction at time=24 hours.

FIG. 5C shows RP-HPLC-MS analyses of a 100 kg refold reaction at time=48 hours.

FIG. 5D shows RP-HPLC-MS analyses of a 100 kg refold reaction at time=70 hours.

FIG. 6 shows a Western Blot demonstrating the presence of a refolded anti-CD22 antibody fragment.

### DETAILED DESCRIPTION OF PREFERRED EMBODIMENTS

It should be appreciated that the particular implementations shown and described herein are examples and are not intended to otherwise limit the scope of the application in any way.

The published patents, patent applications, websites, company names, and scientific literature referred to herein are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

As used in this specification, the singular forms “a,” “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Technical and scientific terms used herein have the meaning commonly understood by one of skill in the art to which the present application pertains, unless otherwise defined. Reference is made herein to various methodologies and materials known to those of skill in the art. Standard reference works setting forth the general principles of recombinant DNA technology include Sambrook et al., “Molecular Cloning: A Laboratory Manual,” 2nd Ed., Cold Spring Harbor Laboratory Press, New York (1989); Kaufman et al., Eds., “Handbook of Molecular and Cellular Methods in Biology in Medicine,” CRC Press, Boca Raton (1995); and McPherson, Ed., “Directed Mutagenesis: A Practical Approach,” IRL Press, Oxford (1991), the disclosures of each of which are incorporated by reference herein in their entireties.

In embodiments, methods are provided for purifying recombinant proteins, including antibodies and antibody fragments. In an embodiment, methods for purifying a recombinant protein from a mixture comprising the recombinant protein and inclusion bodies are provided. The methods suitably comprise solubilizing the mixture comprising the recombinant protein with associated inclusion bodies with a solubilization buffer, clarifying the recombinant protein from the solubilized mixture with one or more depth filters and recovering the clarified recombinant protein. In embodiments, the method does not include centrifuging the solubilized mixture of recombinant protein and inclusion bodies prior to the clarifying the recombinant protein.

As used herein the term “purify” is used to refer to a process by which the desired recombinant protein or proteins are removed from other proteins or undesired products or structures such that the desired protein or proteins are at least about 75% free of other products, at least about 80% free of other products, at least about 90% free of other products, more suitably at least about 95% free of other products, and most suitably at least about 98% of other products.

As used herein, “recombinant proteins” refers to peptides, polypeptides or proteins produced using any suitable expression systems including both prokaryotic and eukaryotic expression systems or using phage display methods (see, e.g., Dower et al., WO91/17271 and McCafferty et al., WO92/01047; and U.S. Pat. No. 5,969,108, which are herein incorporated by reference in their entirety). It should be understood that the term “protein” and proteins” are utilized interchangeably throughout.

As used herein the term “peptide” or “polypeptide” refers to a polymer formed from the linking, in a defined order, of preferably, α-amino acids, D-,L-amino acids, and combinations thereof. The link between one amino acid residue and the next is referred to as an amide bond or a peptide bond. Proteins are polypeptide molecules having multiple polypeptide subunits. The distinction is that peptides are generally short and polypeptides/proteins are generally longer amino acid chains. The term “protein” is intended to also encompass derivatized molecules such as glycoproteins and lipoproteins as well as lower molecular weight polypeptides. “Amino acid sequence” and like terms, such as “polypeptide” or “protein”, are not meant to limit the indicated amino acid sequence to the complete, native amino acid sequence associated with the recited protein molecule.

Methods of producing recombinant proteins using various host cells such as bacteria, plant, yeast, insect and mammalian cells are well known. For example, numerous expression systems are available for expression of proteins utilizing Escherichia coli (E. coli), other bacterial hosts, yeast, and various higher eukaryotic cells such as, for example, COS, CHO, HeLa and myeloma cell lines.

Briefly, the expression of natural or synthetic nucleic acids encoding the desired proteins is generally achieved by operably linking the DNA or cDNA to a promoter (which is either constitutive or inducible), followed by incorporation into an expression cassette. The cassettes can be suitable for replication and integration in either prokaryotic or eukaryotic cell lines. Typical expression cassettes contain transcription and translation terminators, initiation sequences, and promoters useful for regulation of the expression of the DNA encoding the protein. To obtain high level expression of a cloned gene, it is desirable to construct expression, cassettes which contain, at the minimum, a strong promoter to direct transcription, a ribosome binding site for translational initiation, and a transcription/translation terminator. For E. coli, this includes a promoter such as the T7, trp, lac, or lambda promoters, a ribosome binding site and a transcription termination signal. For eukaryotic cells, the control sequences can include a promoter and an enhancer derived from immunoglobulin genes, SV40, cytomegalovirus, and a polyadenylation sequence, and may include splice donor and acceptor sequences. The cassettes can be transferred into the chosen host cell by well-known methods such as calcium chloride transformation or electroporation for E. coli and calcium phosphate treatment, or electroporation or lipofection for mammalian cells. Cells transformed by the cassettes can be selected by resistance to antibiotics conferred by genes contained in the cassettes, such as the amp, gpt, neo and hyg genes.

Following expression in the host cell, the recombinant proteins are often associated with inclusion bodies from which they must be extracted prior to being refolded. In the present methods, a mixture comprising the desired recombinant protein or proteins with associated inclusion bodies is solubilized with a solubilization buffer.

Examples of solubilization buffers include those known in the art. For example, suitable solubilization buffers comprise a reducing agent to separate disulfide bonds. Exemplary buffers can comprise Tris, guanidine, EDTA and DTE. In embodiments, the solubilization buffer comprises ethanolamine, arginine, EDTA, urea and DTE. For example, the solubilization buffer can comprise about 20 mM to about 70 mM ethanolamine, about 200 mM to about 2 M arginine, about 1 mM to about 3 mM EDTA, about 5 M to about 10 M urea and about 5 mM to about 20 mM DTE, and can have a pH of about 10 to about 11. Suitably, the solubilization buffer comprises about 30 mM to about 60 mM ethanolamine, about 200 mM to about 1 M arginine, about 1 mM to about 3 mM EDTA, about 6 M to about 9 M urea and about 8 mM to about 15 mM DTE. More suitably, the solubilization buffer comprises about 40 mM to about 60 mM ethanolamine, about 400 mM to about 600 mM arginine, about 1 mM to about 3 mM EDTA, about 7 M to about 9 M urea and about 8 mM to about 12 mM DTE. Most suitably the solubilization buffer comprises about 50 mM ethanolamine, about 500 mM arginine, about 2 mM EDTA, about 8 M urea and about 10 mM DTE, and has a pH of about 10.5.

Following solubilization of the mixture comprising the desired recombinant protein or proteins and inclusion bodies, the recombinant protein or proteins are clarified from the solubilized mixture with one or more depth filters, suitably two or more depth filters.

As used herein, the term “clarify” or “clarifying” refers to the separation of the desired, recombinant protein from the solubilized mixture by removing the desired protein from unwanted proteins and other material, wherein the unwanted material is retained on a clarifying medium (e.g., a filter). As discussed in the background section, clarification typically involves centrifuging a solubilized mixture of recombinant protein and inclusion bodies, followed by depth filtration, or depth filtration followed by tangential flow filtration, or just tangential flow filtration. It has been surprisingly found that clarification of the recombinant protein can be performed by passing the solubilized mixture through one or more depth filters. Thus, advantageously, the methods do not include centrifuging the solubilized mixture of recombinant protein and inclusion bodies prior to the clarification. In addition, the methods suitably do not include the use of tangential flow filtration following the depth filtration.

In embodiments, the methods specifically exclude centrifuging the solubilized mixture of recombinant protein and inclusion bodies prior to the clarification. It has been determined that excluding a centrifugation step materially affects the basic and novel characteristics of the disclosed methods. These basic and novel characteristics include, but are not limited to, any of the following: omission of the time typically associated with centrifugation; omission of the energy cost typically associated with centrifugation; omission of the equipment costs typically associated with centrifugation; and omission of the process inefficiency typically associated with centrifugation. In addition, removing the centrifugation step allows for scale-up in instances where a centrifuge is not available, for example, in a small Good Manufacturing Practice (GMP) purification site.

Thus, in suitable embodiments, methods are provided for purifying a recombinant protein from a mixture comprising the recombinant protein and inclusion bodies, the method consisting essentially of solubilizing the mixture comprising the recombinant protein with associated inclusion bodies with a solubilization buffer, clarifying the recombinant protein from the solubilized mixture with one or more depth filters and recovering the clarified recombinant protein. The addition of a centrifugation step prior to the clarifying step is considered a material alteration to such methods and is thus excluded from such methods that consist essentially of the recited steps.

In still further embodiments, methods are provided for purifying a recombinant protein from a mixture comprising the recombinant protein and inclusion bodies, the method consisting of solubilizing the mixture comprising the recombinant protein with associated inclusion bodies with a solubilization buffer, clarifying the recombinant protein from the solubilized mixture with one or more depth filters and recovering the clarified recombinant protein. In such embodiments, no additional steps other than the recited steps are permitted.

Omission of centrifugation from the clarification of the methods described herein provided unexpected and surprising results, as the desired recombinant proteins were be recovered in amounts and purities that were at least as high as methods that utilized centrifugation, while providing the basic and novel characteristics identified above. It was not expected that omission of centrifugation would allow for sufficient clarification utilizing only depth filtration.

As used herein, “depth filtration” refers to filtration utilizing filters that comprise a porous filtration medium that allows retention of particles throughout the medium, rather than just on the surface of the medium. Exemplary depth filtration media and filtration methods are described herein or otherwise known in the art. Following the depth filtration, the clarified recombinant protein is recovered.

In exemplary embodiments, the methods are utilized to purify a recombinant protein that is an antibody or an antibody fragment. As used herein, the term “antibody” or “antibodies” refers to all types of immunoglobulins, including IgG, IgM, IgA, IgD, and IgE, including binding fragments thereof (i.e., fragments of an antibody that are capable of specifically binding to the antibody's target molecule, such as Fab, and F(ab′)₂ fragments), as well as recombinant, humanized, polyclonal, and monoclonal antibodies and/or binding fragments thereof. The term antibody also includes genetically engineered forms such as chimeric antibodies (e.g., humanized murine antibodies), heteroconjugate antibodies (e.g., bispecific antibodies), recombinant single chain Fv fragments (scFv), and disulfide stabilized (dsFv) Fv fragments.

Typically, an antibody has a heavy and light chain. Each heavy and light chain contains a constant region and a variable region, (the regions are also known as “domains”). Light and heavy chain variable regions contain a “framework” region interrupted by three hypervariable regions, also called “complementarity-determining regions” or “CDRs.” The extent of the framework region and CDRs have been defined. The sequences of the framework regions of different light or heavy chains are relatively conserved within a species. The framework region of an antibody, that is the combined framework regions of the constituent light and heavy chains, serves to position and align the CDRs in three dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is located in the variable domain of the light chain of the antibody in which it is found.

References to “V_{H}” or a “VH” refer to the variable region of an immunoglobulin heavy chain, including an Fv, scFv, dsFv or Fab. References to “V_{L}” or a “VL” refer to the variable region of an immunoglobulin light chain, including of an Fv, scFv, dsFv or Fab.

The term “single chain Fv” or “scFv” refers to an antibody in which the variable domains of the heavy chain and of the light chain of a traditional two chain antibody have been joined to form one chain. Typically, a linker peptide is inserted between the two chains to allow for proper folding and creation of an active binding site. The term “linker peptide” includes reference to a peptide within an antibody binding fragment (e.g., Fv fragment) which serves to indirectly bond the variable domain of the heavy chain to the variable domain of the light chain.

Suitably, the mixture that is solubilized and clarified comprises an antibody fragment, such as a heavy chain (V_{H}) antibody fragment or a light chain (V_{L}) antibody fragment with associated inclusion bodies. In additional embodiments, the mixture can comprise both V_{H} antibody fragments and V_{L} antibody fragments associated with inclusion bodies. When both V_{H} antibody fragments and V_{L} antibody fragments are present in the mixture, the ratio of the V_{H} antibody fragments to the. V_{L} antibody fragments is suitably tailored so as to maximize formation of the final antibody fragment. Suitably, V_{H} antibody fragments and V_{L} antibody fragments are present at an initial molar ratio of about 1:1 to about 1:20 (V_{H} to V₁), or about 20:1 to about 1:1, more suitably about 1:1 to about 1:10, or about 10:1 to about 1:1, or most suitably about 1:1.

In exemplary embodiments, the V_{H} antibody fragment is an anti-CD22 V_{H} antibody fragment and the V_{L} antibody fragment is an anti-CD22 V_{L} antibody fragment. Suitably, the antibody fragments are anti-CD22 fragments as disclosed in the '012 patent. The anti-CD22 antibodies disclosed in the '012 patent have a variable light chain having the sequence of antibody RFB4 and a variable heavy chain having the sequence of antibody RFB4, but in which residues 100, 100A and 100B of CDR3 of the V_{H} chain (as numbered by the Kabat and Wu numbering system) have an amino acid sequence selected from the group consisting of: THW, YNW, TTW, and STY. As described herein, solubilization and clarification of the V_{H} and V_{L} portions of the anti-CD22 antibody in a mixture allow for subsequent refolding into a functional and active anit-CD22 antibody fragment.

As set forth in the '012 patent, these anti-CD22 antibody fragments can be conjugated to various therapeutic agents, including cytotoxic agents, for delivery to specific cells targeted by the anti-CD22 antibody fragments. The term “therapeutic agent” includes any number of compounds currently known or later developed to act as anti-neoplastic, anti-inflammatories, cytokines, anti-infectives, enzyme activators or inhibitors, allosteric modifiers, antibiotics or other agents administered to induce a desired therapeutic effect in a patient. The therapeutic agent may also be a toxin or a radioisotope, where the therapeutic effect intended is, for example, the killing of a cancer cell. Exemplary toxins include ricin, abrin, diphtheria toxin and subunits thereof, as well as botulinum toxins A through F. Some of these exemplary toxins are available from commercial sources (e.g., Sigma Chemical Company, St. Louis, Mo.).

In exemplary embodiments, the toxin is Pseudomonas exotoxin (PE). The term “Pseudomonas exotoxin” as used herein refers to a full-length native (naturally occurring) PE or a PE that has been modified. Such modifications may include, but are not limited to, elimination of domain la, various amino acid deletions in domains Ib, II and III, single amino acid substitutions and the addition of one or more sequences at the carboxyl terminus.

As described herein, suitably the methods utilize one or more depth filters for the clarification of the recombinant protein. In exemplary embodiments, the recombinant protein is clarified with two or more, three or more, four or more, five or more, six or more, seven or more, eight or more, nine or more, ten or more, etc, depth filters. Clarification with two or more depth filters suitably comprises, passing the solubilized mixture through a first depth filter followed by further clarifying by passing the permeate including the recombinant protein from the first depth filter through a second depth filter (and further depth filters as desired).

In embodiments, the clarifying comprises passing the solubilized mixture through a first depth filter comprising cellulose fiber and diatomaceous earth. Additional filter media for the depth filter can also be utilized. Suitably, the first depth filter has a nominal micron rating of about 0.1 μm to about 1 μm. The clarifying then further comprises clarifying with a second depth filter. That is passing the permeate from the first depth filter through a second depth filter. Suitably, the second depth filter comprises cellulose fiber and diatomaceous earth. Additional filter media for the depth filter can also be utilized. Suitably, the second depth filter has a nominal micron rating of less than about 0.1 μm. Additional depth filters can also be utilized, where the permeate from the 2^{d} depth filter is passed through a 3^{rd} depth filter, and so forth. Exemplary depth filters are readily available and can be purchased, for example, from MILLIPORE™, Billerica, Mass. (MILLISTAK+™); Pall Corporation, Port Washington, N.Y. (STAX™); 3M Purification Inc. (CUNO®), Meriden, Conn. (ZETA PLUS™); and BEGEROW, Langenlonsheim, Germany.

Exemplary CUNO® filters include ZETA PLUS™ filters 10SP05A, 30SP10A, 60SP30A and 90SP60A. Flow rates of approximately 0.1 L/min/ft² to about 0.5 L/min/ft² (e.g., about 0.2 L/min/ft²) can be utilized, with a pressure of about 15-40 pounds/in² (PSI) (e.g., about 25 PSI) and an output of about 2-10 L/ft² (e.g., about 5 L/ft²), with such filters.

In suitable embodiments, the methods utilize two depth filters, for example, a first depth filter that is a MILLIPORE™ C0HC depth filter and a second depth filter that is a MILLIPORE™ X0HC depth filter. The depth filters are suitably utilized as part of a MILLISTAK+™ Pod disposable depth filter system to provide scalable filtration. Suitably, the C0HC depth filter will have a surface area of about 7-8 m² (e.g., 7.70 m²), and the X0HC depth filter will have a surface area of about 5-6 m² (e.g., 5.50 m²). Flowrates and fluid flux through the depth filters can be determined and modified by those of ordinary skill in the art. Generally, the flowrates for the clarification through the depth filters will be about 5 to about 20 L/min, more suitably about 6 to about 16 L/min. Flux through the depth filters will generally range from about 40 to about 150 L/m²/hour (LMH) for the C0HC filters and about 60 to about 200 LMH for the X0HC filters, though other flowrates and fluxes can also be utilized.

The methods suitably provide yields of the clarified recombinant protein of greater than about 50%, suitably greater than about 60%, greater than about 70%, greater than about 80%, greater than about 85%, greater than about 90%, greater than about 95% or greater than about 98%. The protein yield is calculated by measuring the amount of recombinant protein recovered from the clarification as compared to the recombinant protein present prior to the clarification.

The clarification methods suitably comprise clarifying a mixture of V_{H} and V_{L} antibody fragments, including anti-CD22 V_{H} and V_{L} antibody fragments. In other embodiments, the V_{H} and V_{L} fragments can be clarified separately (e.g., after separate solubilization) and then combined prior to refolding, as discussed herein. Suitably, the V_{H} and V_{L} antibody fragments can be kept separate throughout the purification process until refolding, thereby allowing for a greater yield of final, folded antibody fragment.

In embodiments, the methods further comprise concentrating the clarified recombinant protein and refolding the clarified recombinant, protein in a protein refolding buffer. Suitably, the concentrating comprises concentrating using tangential flow filtration to a concentration of about 1 mg/mL to about 10 mg/mL total protein, suitably about 5 mg/mL total protein.

In exemplary embodiments, the refolding of the clarified recombinant protein comprises diluting the concentrated, clarified recombinant protein in a protein refolding buffer. Suitable protein refolding buffers that can be utilized are known in the art and suitably comprise Tris, L-arginine, oxidized glutathione (GSSG) and EDTA. For example, a suitable refolding buffer can comprise about 50 mM to about 200 mM Tris (e.g., about 100 mM) at pH 8, about 100 mM to about 1 M L-arginine (e.g., about 500 mM), about 5 mM to about 10 mM GSSG (e.g., about 8 mM) and about 0.5 mM to about 4 mM EDTA (e.g., about 2 mM).

An additional suitable protein refolding buffer comprises about 20 mM to about 70 mM ethanolamine; about 0.5 M to about 2 M arginine, about 0.5 mM to about 3 mM EDTA, and about 0.5 mM to about 1.5 mM GSSG, and has a pH of about 9 to 10.

Following the dilution with the protein refolding buffer, the diluted, clarified recombinant protein is incubated at a pH of about 9 to about 10 and at a temperature of about 2° C. to about 15° C., for about 48 hours to about 96 hours. The folded, recombinant protein is then recovered. It has been determined that a refolding buffer having a pH of about 9 to about 10 produces more refolded final protein than a buffer having a pH of about 8 or less.

In the case of an antibody fragment comprising separate V_{H} and V_{L} portions, diluting a mixture that comprises both the V_{H} and V_{L} portions together in a suitable protein refolding buffer allows the V_{H} and V_{L} portions to refold into a functional antibody fragment comprising both V_{H} and V_{L} portions. For example, V_{H} and V_{L} portions of an anti-CD22 antibody fragment can be mixed together, clarified and then refolded to form a fully functional anti-CD22 antibody fragment. Alternatively, the VH and VL fragments can be separately clarified prior to mixing and refolding into a functional antibody fragment, allowing further modification and purification of the antibody fragment. For example, the anti-CD22 antibody can be further conjugated to a therapeutic agent, including a cytotoxic agent, as described herein and in the '012 patent, the disclosure of which is incorporated herein by reference.

As described herein, the methods suitably comprise refolding the recombinant protein. Thus, in additional embodiments, a protein refolding buffer is provided for refolding a solubilized recombinant protein, including antibody fragments, such as anti-CD22 antibody fragments. The protein refolding buffer suitably comprises about 20 mM to about 70 mM ethanolamine; about 500 mM to about 2 M arginine; about 0.5 mM to about 3 mM EDTA; and about 0.5 mM to about 1.5 mM GSSG. Suitably the refolding buffer has a pH of about 9 to about 10. In exemplary embodiments, the protein refolding buffer comprises about 30 mM to about 60 mM ethanolamine; about 800 mM to about 1.5 M arginine; about 1 mM to about 3 mM EDTA; and about 0.7 mM to about 1.2 mM GSSG. In further embodiments, the protein refolding buffer comprises about 50 mM ethanolamine; about 1 M arginine; about 2 mM EDTA; and about 0.9 mM GSSG. Suitably, the refolding buffer has a pH of about 9.5.

It has been surprisingly found that use of the refolding buffers reduce or eliminate the presence of a glutathione adduct of the recombinant proteins, particularly the glutathione adduct of an antibody fragment such as an anti-CD22 antibody fragment. Suitably, the refolding buffers reduce the presence of a glutathione adduct of a recombinant protein such as an antibody fragment such that the final refolded antibody fragment comprises less than about 40% of the glutathione adduct of the antibody fragment. More suitably, the refolding buffers reduce the glutathione adduct to less than about 30%, less than about 20%, less than about 15%, less than about 10%, less than about 5%, less than about 2%, less than about 1%, or less than about 0.1% of the recovered recombinant protein (e.g., antibody fragment).

In still further embodiments, a refolding buffer is provided that consists essentially of about 20 mM to about 70 mM ethanolamine; about 500 mM to about 2 M arginine; about 0.5 mM to about 3 mM EDTA; and about 0.5 mM to about 1.5 mM GSSG. Such buffers exclude additional . components that materially affect the basic and novel characteristics of the buffers, specifically ability of the buffer to reduce the glutathione adduct of a recombinant protein to less than about 30%. In still further embodiments, refolding buffers are provided that consist of about 20 mM to about 70 mM ethanolamine; about 500 mM to about 2 M arginine; about 0.5 mM to about 3 mM EDTA; and about 0.5 mM to about 1.5 mM GSSG, and thus exclude other components (but can include water for dilution as necessary).

In additional embodiments, methods of refolding a solubilized recombinant protein are provided. Such methods suitably comprise concentrating a solubilized recombinant protein and diluting the concentrated recombinant protein in a refolding buffer. As described herein, suitably the refolding buffer comprises about 20 mM to about 70 mM ethanolamine, about 500 mM to about 2 M arginine, about 0.5 mM to about 3 mM EDTA, and about 0.5 mM to about 1.5 mM GSSG.

The diluted recombinant protein is incubated at a pH of about 9 to about 10 and at a temperature of about 2° C. to about 15° C., for about 48 hours to about 96 hours. The refolded recombinant protein is then recovered.

As described herein, suitably the solubilized recombinant protein comprises recombinant antibodies or recombinant antibody fragments. In exemplary embodiments, the recombinant antibody fragments comprise recombinant V_{H} antibody fragments and recombinant V_{L} antibody fragments, such as recombinant anti-CD22 V_{H} antibody fragments and recombinant anti-CD22 V_{L} antibody fragments. Suitably, the V_{H} and V_{L} fragments are mixed prior to the refolding.

The use of the refolding buffers described herein suitably reduce the glutathione adduct of the recovered protein, such that less than about 30% of the recovered, refolded recombinant protein is a glutathione adduct of the recombinant protein. More suitably less than about 20%, less than about 10%, less than about 5%, less than about 2%, less than about 1% or less than about 0.1% of the recovered refolded recombinant protein is a glutathione adduct of the recombinant protein.

In further embodiments, methods of producing a recombinant antibody fragment comprising a V_{H} antibody fragment and a V_{L} antibody fragment are provided. The methods suitably comprise expressing a polynucleotide encoding a V_{H} antibody fragment in a first bacterial cell and expressing a polynucleotide encoding a V_{L} antibody fragment in a second bacterial cell. In further embodiments, the V_{H} and the V_{L} fragment can be produced in the same bacterial cell. Alternatively, the V_{H} and the V_{L} fragments can be produced in different types of cells (e.g., mammalian or bacterial), or can be produced together in a cell other than a bacterial cell.

As described herein, methods of producing recombinant proteins are well known in the art, and suitably comprise expressing polynucleotides encoding various proteins in a host cell. As used herein, bacterial cells include any bacterial cell that can be utilized in the production of recombinant proteins, including E. coli.

The V_{H} antibody fragment and the V_{L} antibody fragment are mixed to generate a mixture, wherein the mixture further comprises inclusion bodies. In embodiments where the V_{H} and the V_{L} are produced in the same cell, mixing them together is not required. As described herein, production of recombinant proteins using bacterial cells produces the desired proteins associated with inclusion bodies. The desired proteins are suitably clarified from the inclusion bodies prior to further folding and purification.

The mixture comprising the V_{H} antibody fragment, the V_{L} antibody fragment with associated inclusion bodies is solubilized with a solubilization buffer. Exemplary solubilization buffers for use in the methods are described herein, and suitably comprise ethanolamine, arginine, EDTA, urea and DTE, for example, about 20 mM to about 70 mM ethanolamine, about 200 mM to about 2 M arginine, about 1 mM to about 3 mM EDTA, about 5 M to about 10 M urea and about 5 mM to about 20 mM DTE. The solubilization buffer suitably has a pH of about 10 to about 11, for example about pH 10.5.

The V_{H} antibody fragment and the V_{L} antibody fragment are clarified from the solubilized mixture with one or more depth filters. As described herein, the methods do not include centrifuging the solubilized mixture of V_{H} antibody fragment, V_{L} antibody fragment and inclusion bodies. Exclusion of a centrifugation step materially affects the novel and basic characteristics of the methods, as described herein.

The clarified V_{H} antibody fragment and the clarified V_{L} antibody fragment are recovered and then concentrated. The concentrated, clarified V_{H} antibody fragment and the concentrated, clarified V_{L} antibody fragment are diluted with a refolding buffer. As described herein, in suitable embodiments, the refolding buffer comprises about 20 mM to about 70 mM ethanolamine, about 500 mM to about 2 M arginine, about 0.5 mM to about 3 mM EDTA, and about 0.5 mM to about 1.5 mM GSSG and has a pH of about 9-10, suitably about 9.5.

The diluted, clarified V_{H} antibody fragment and the diluted, clarified V_{L} antibody fragment are incubated at a pH of about 9 to about 10 and at a temperature of about 2° C. to about 15° C., for about 48 hours to about 96 hours. This allows for the V_{H} antibody fragment and the V_{L} antibody fragment to refold into a fully functional and active antibody fragment. The antibody fragment is then recovered, e.g., via art-known methods to recover and store the antibody fragment if desired.

As described herein, suitably the V_{H} antibody fragment is an anti-CD22 V_{H} antibody fragment, the V_{L} antibody fragment is an anti-CD22 V_{L} fragment, and the recombinant antibody fragment is an anti-CD22 antibody fragment, such as those disclosed in the '012 patent. As described herein, these antibody fragments can be further conjugated to a therapeutic or toxic agent.

The V_{H} antibody fragment and the V_{L} antibody fragment are suitably clarified with two or more depth filters, and in exemplary embodiments with two depth filters. As described herein, centrifugation is not utilized prior to clarification using depth filtration.

In embodiments, a first depth filter that is utilized in the clarification comprises cellulose fiber and diatomaceous earth, and has a nominal micron rating of about 0.1 μm to about 1 μm (e.g., a C0HC depth filter). The methods suitably further comprise clarifying the V_{H} antibody fragment and the V_{L} antibody fragment with a second depth filter, the second depth filter comprising cellulose fiber and diatomaceous earth, and wherein the second depth filter has a nominal micron rating of less than about 0.1 μm (e g, an X0HC depth filter).

As described herein, has been determined that excluding a centrifugation step materially affects the basic and novel characteristics of the disclosed methods. Thus, in embodiments, methods are provided that consist essentially of the recited steps, or in further embodiment, consist of the recited steps, specifically excluding a centrifugation step prior to clarification with depth filtration.

The methods described herein can be readily scaled to large manufacturing capacities. For example, the methods can be readily scaled to utilize volumes on the order of about 100 L to about 20,000L, suitably about 100 L to about 10,000L, about 100 L to about 5,000 L, about 500 L to about 1,000 L, e.g., about 500 L, about 600 L, about 700 L, about 800 L, about 900 L or about 1,000 L.

It will be readily apparent to one of ordinary skill in the relevant arts that other suitable modifications and adaptations to the methods and applications described herein can be made without departing from the scope of any of the embodiments. The following examples are included herewith for purposes of illustration only and are not intended to be limiting.

### EXAMPLES

### Example 1

### Anti-CD22 Antibody Fragment Purification

### Solubilization

V_{H} and V_{L} portions of an anti-CD22 antibody fragment were produced via recombinational cloning methods in E. coli. Once the desired refold mass was determined, inclusion bodies were mixed such that for each kg of refold mass, 0.56 g of V_{H} and 0.128 g of V₁ would be reacted. The addition of 4.375 g of V_{H} for every 1 g of V_{L} is equivalent to a 1:1 molar ratio. For example, a 10 kg refold requires 5.6 g of V_{H} and 1.28 g of V_{L}. The addition of 5.6 g of V_{H} is made by the addition of 181.60 g of V_{H} solution because the inclusion bodies are 65% pure and contain 0.16582 g of protein per gram. Similarly, the addition of 1.28 g of V_{L} is made by the addition of 125.44 g of V_{L} solution. The mixture of V_{H} of and V_{L}, respectively of 26.91% and 20% solids, was then diluted with TE buffer (50 mM Tris, 20 mM EDTA pH 7.5) to create a mixture of 15% solids. The 15% inclusion bodies mixtures were each solubilized with 5 volumes of solubilization buffer (50 mM ethanolamine, 0.5 M arginine, 2 mM EDTA, 8M urea, and 10 mM DTE). Each solubilization reaction yielded refold values ranging from 40.5 to 49.17 μg/mL. Inclusion bodies mixtures have been solubilized under similar conditions for multiple lots and various scales. Table 1 below presents a summary of the solubilization conditions utilized.

TABLE 1

| | | |
|---|---|---|
| Desired Refold Size (kg) | 1.25 | 10 |
| V_{H} Lot | A | B |
| V_{H} Percent Solids | 20.90% | 26.91% |
| V_{H} g Protein/g IB | 0.129 | 0.16852 |
| V_{H} IB % Purity | 65% | 68% |
| V_{H} Mass of Chain (g) | 0.7 | 5.6 |
| V_{H} Biomass Required (g) | 8.35 | 48.87 |
| V_{H} Slurry Required (g) | 39.94 | 181.60 |
| V_{L} Lot | C | D |
| V_{L} Percent Solids | 26% | 20% |
| V_{L} g Protein/g IB | 0.1248 | 0.11596 |
| V_{L} IB % Purity | 51% | 0.44 |
| V_{L} Mass of Chain (g) | 0.16 | 1.28 |
| V_{L} Biomass Required (g) | 2.51 | 25.09 |
| V_{L} Slurry Required (g) | 9.67 | 125.44 |
| TE Required (g) | 22.79 | 186.0 |
| Mass of 15% Slurry (g) | 72.4 | 493.04 |
| Solubilization Buffer (g) | 387.10 | 2465.18 |
| Solubilized Reaction (g) | 459.5 | 2958.21 |
| Refold Yield (μg/mL) | 49.17 | 40.5 |

Table 2 outlines solubilization conditions for two lots of inclusion body mixtures. The amounts of V_{H} and V_{L} were reduced by half so that the total volume and total protein in the reaction would be equal for both lots. Therefore, for each kg of desired refold, 0.28 g of V_{H} and 0.064g of V_{L} were mixed and solubilized. Comparison of the 5 kg refold reaction in Table 2 to the 5 kg reaction in Table 1 shows that although there is half the amount of V_{H} and V_{L} in the solubilization reaction, the total mass of each solubilized material is similar, 2070.20 g compared to 2958.21 g. Both reactions contained similar total protein levels, were clarified by depth filters of the same membrane areas, and refolded in the same volume. Although the impact on these downstream unit operations was minimal, the refold yields were different. The decrease in concentration of V_{H} (from 0.56 g to 0.28 g per kg of refold) and V_{L} (from 0.128 g to 0.064g per kg of refold) in the solubilization reaction decreased the concentration of the anti-CD22 antibody fragment formed in the refold from 49.17 μmg/mL to 29 μg/mL. The decrease in the yield was noted in the large scale solubilization reaction as well. The addition of half of the mass of V₁₄ and V_{L} in the solubilization reaction resulted in half the amount of anti-CD22 antibody fragment formed in the refold reaction. Therefore, twice the amount of refolds and purifications would need to be performed.

TABLE 2

| | | |
|---|---|---|
| Desired Refold Size (kg) | 5 | 100 |
| V_{H} Lot | E | F |
| V_{H} Percent Solids | 19.40% | 19.40% |
| V_{H} (mg/mL) | 7 | 7 |
| V_{H} IB % Purity | 0.35 | 0.35 |
| V_{H} Mass of Chain (g) | 1.4 | 28.0 |
| V_{H} Slurry Required (g) | 200.00 | 4000.0 |
| V_{L} Lot | G | H |
| V_{L} Percent Solids | 28.4% | 28.4% |
| V_{L} (mg/mL) | 7 | 7 |
| V_{L} IB % Purity | 0.16 | 0.16 |
| V_{L} Mass of Chain (g) | 0.32 | 6.40 |
| V_{L} Slurry Required (g) | 45.71 | 914.29 |
| TE Required (g) | 99.50 | 1990.1 |
| Mass of 15% Slurry (g) | 345.22 | 6904.38 |
| Solubilization Buffer (g) | 1728.00 | 34200 |
| Solubilized Reaction (g) | 2070.20 | 41014.3 |
| Refold Yield (μg/mL) | 29.0 | 22.8 |

### Clarification

For comparison purposes, clarification utilizing centrifugation followed by depth filtration was carried out. Solubilized inclusion bodies were fed to a disc stack centrifuge at 129 mL/min, and centrifuged at 9470 rpm under a pressure of 30 psi. The centrate was still turbid and required further clarification. The centrate was depth filtered through a BEGEROW PR Steril S 100 filter with a capacity of 28 L/m² (BEGEROW, Catalog # S001P). The combination of the centrifugation and depth filtration resulted in material with a turbidity of 2 ntu. Table 3 outlines the parameters for the clarification. Heavy and light chain containing inclusion bodies lots were mixed, solubilized, and clarified using centrifugation followed by depth filtration. The clarified material was successfully refolded in a reaction that produced 40.5 μg/mL anti-CD22 antibody fragment.

TABLE 3

Creation of 15% Inclusion Bodies

| | Heavy | Light |
|---|---|---|
| | Chain Biomass | Chain Biomass |
| Lot | I | J |
| Percent Solids | 26.91% | 20% |
| g Protein/g IB | 0.16852 | 0.11596 |
| IB % Purity | 0.68 | 0.44 |
| Mass of Chain (g) | 5.6 | 1.28 |
| Slurry Required (g) | 181.60 | 125.44 |
| TE Required (g) | 144.19 | 41.81 |

| | |
|---|---|
| | Solubilization |
| Mass of 15% Slurry (g) | 493.04 |
| Solubilization Buffer (g) | 2465.18 |
| Solubilized Reaction (g) | 2958.21 |
| Incubation Time (hr) | 2 |
| Incubation Temperature | 20-25 |
| (° C.) | |
| Mixing | Stir Plate |
| Solubilization V_{H} (mg/mL) | 1.31 |
| Solubilization V_{H} (mg) | 3866.01 |
| Solubilization V_{L} (mg/mL) | 0.36 |
| Solubilization V_{L} (mg) | 1064.96 |
| | Clarification - Centrifugation |
| Starting Turbidity (ntu) | 222 |
| Centrifugation Force (g) | 12,000 |
| Centrifugation Time (hr) | 2 |
| Centrate Temp (° C.) | 4 |
| Mass of Centrate (g) | 3027.7 |
| Turbidity of Centrate (ntu) | 102 |
| Centrate V_{H} (mg/mL) | 1.26 |
| Centrate V_{H} (mg) | 3814.90 |
| Centrate V_{L} (mg/mL) | 0.35 |
| Centrate V_{L} (mg) | 1059.70 |
| Centrifugation V_{H} yield (%) | 98.68 |
| Centrifugation V_{L} yield (%) | 99.51 |
| | Clarification - Depth Filtration |
| Depth Filter Manufacturer | Begerow |
| Membrane Model | S001P102 |
| Membrane Area (cm²) | 160 |
| Filtrate turbidity (ntu) | 1.75 |
| Filtrate mass (g) | 2634.4 |
| Filtrate V_{H} (mg/mL) | 1.01 |
| Filtrate V_{H} (mg) | 2660.74 |
| Filtrate V_{L} (mg/mL) | 0.31 |
| Filtrate V_{L} (mg) | 816.66 |
| Filtrate V_{H} yield (%) | 69.75 |
| Filtrate V_{L} yield (%) | 77.07 |
| Clarified V_{H} yield (%) | 68.82 |
| Clarified V_{L} yield (%) | 76.69 |
| Refold Yield (μg/mL) | 40.5 |

TFF membranes were also evaluated for clarification. The capacities of the 0.1 μm and 1000 KDa membranes were too low to process the expected 150 L volume of the manufacturing process. Therefore, although the V_{H} and V_{L} recoveries of clarification across the 0.1 μm membrane were comparable to the combination of centrifugation and depth filtration (76.34% and 80.57% respectively), TFF was not deemed to be acceptable.

Anti-CD22 antibody fragment was also clarified using only depth filtration (no centrifugation step prior to clarification by depth filtration) and also with tangential flow filtration (TFF), for comparison purposes. Inclusion bodies (V_{H} and V_{L} lots) were mixed and solubilized as discussed above. The solubilized inclusion bodies were then clarified using the membranes in Table 4. Solubilized material was initially filtered through the C0HC depth filter. Passage of the material through the depth filter reduced the turbidity to 68.5 ntu. This filtrate was then further clarified through the tighter A1HC and X0HC filters. The pressure rapidly increased in the A1HC depth filter, resulting in an unacceptable capacity of 2.9 L/m². The steep pressure increase was not seen during filtration of the C0HC filtrate across the X0HC filter, as a capacity of 129.5 L/m² was recorded. The C0HC and X0HC filter demonstrated good throughput as well turbidity of the material. The V_{H} and V_{L} recoveries were 74.82% and 68.63%, respectively.

TABLE 4

| Filter Evaluated | Capacity (L/m²) | Turbidity (NTU) |
|---|---|---|
| Depth Filters | | |
| C0HC | 61.1 | 68.5 |
| A1HC (using C0HC filtrate) | 2.9 | N/A |
| X0HC (using C0HC filtrate) | 129.5 | 1.51 |
| TFF Membrane | | |
| 0.1 μm Pellicon | 11.0 | 1.7 |
| 1000 kDa Pellicon 2 | 7.3 | 0.72 |

A depth filtration train comprising C0HC and X0HC filters was further evaluated to determine if the process would scale effectively. Table 5 outlines the parameters that were utilized and recorded during bench scale and scale-up runs. Inclusion body mixtures were mixed to a 1:1 molar ratio, diluted to 15% solids, and solubilized. The solubilized material was clarified by the C0HC-X0HC depth filter train. RP-HPLC-MS analysis was used to quantify the yields of V_{H} and V_{L}. The V_{H} and V_{L} recoveries across the filtration operation ranged from 78.6 to 83.3% and 63.9 to 79.8%, respectively. The turbidity of the material ranged from 2.1 to 2.2 mu. The yield of anti-CD22 antibody fragment formed during the refold reaction ranged from 37.8 to 68.1 μg/mL. The consistent recovery of the heavy and light chains, as well as the turbidity and refold yield values across scales, indicated that the C0HC-X0HC depth filter train was superior to the centrifugation/depth filtration procedure.

TABLE 5

| | Creation of 15% Inclusion Body Mixtures | | | |
|---|---|---|---|---|
| | Heavy Chain Biomass | | Light Chain Biomass | |
| Lot | J | | K | |
| Percent Solids | 26.91% | | 20% | |
| g Protein/g IB | 0.16852 | | 0.11596 | |
| IB % Purity | 0.68 | | 0.44 | |
| Refold Size (kg) | 5 | 5 | 5 | 100 |
| Mass of V_{H} Chain (g) | 2.84 | 2.81 | 2.82 | 56 |
| V_{H} Slurry Required (g) | 92.10 | 91.20 | 91.50 | 1815.99 |
| Mass of V_{L} Chain (g) | 0.66 | 0.65 | 0.65 | 12.8 |
| V_{L} Slurry Required (g) | 64.90 | 63.5 | 63.3 | 1254.35 |
| TE Required (g) | 94.00 | 94.50 | 90.8 | 1860.01 |
| Molar ratio (V_{H}:V_{L}) | 1:1 | | | |
| | Solubilization | | | |
| Mass of 15% Slurry (g) | 251.00 | 249.2 | 245.6 | 4930.36 |
| Solubilization Buffer (g) | 1260.4 | 1246 | 1228 | 24651.79 |
| Solubilized Reaction (g) | 1511.4 | 1495.2 | 1473.6 | 29582.15 |
| Incubation Time (hr) | 2 | 2 | 2 | 2 |
| Incubation Temperature | 20-25 | 20-25 | 20-25 | 20-25 |
| (° C.) | | | | |
| Mixing (~120 minutes) | stir plate | stir plate | stir plate | mobius |
| Solubilization V_{H} | 1.73 | 2.42 | 1.78 | 1.3 |
| (mg/mL) | | | | |
| Solubilization V_{H} (mg) | 2614.73 | 3618.38 | 2623.01 | 38456.79 |
| Solubilization V_{L} | 0.4 | 0.61 | 0.61 | 0.4 |
| (mg/mL) | | | | |
| Solubilization V_{L} (mg) | 604.56 | 912.03 | 898.90 | 11832.86 |
| | Clarification - Depth Filtration | | | |
| Depth Filter | Millipore | Millipore | Millipore | Millipore |
| Manufacturer | | | | |
| Membrane Model | C0HC | C0HC | C0HC | C0HC |
| Membrane Area (m²) | 0.054 | 0.054 | 0.054 | 0.77 |
| Membrane Model | X0HC | X0HC | X0HC | X0HC |
| Membrane Area (m²) | 0.027 | 0.027 | 0.027 | 0.55 |
| Filtrate turbidity (ntu) | 2.1 | 2.2 | 2.1 | 2.1 |
| Filtrate mass (g) | 2192.2 | 2420.2 | 2195.7 | 37800 |
| Filtrate V_{H} (mg/mL) | 0.94 | 1.23 | 0.95 | 0.8 |
| Filtrate V_{H} (mg) | 2060.69 | 2976.85 | 2085.92 | 30240 |
| Filtrate V_{L} (mg/mL) | 0.22 | 0.3 | 0.32 | 0.2 |
| Filtrate V_{L} (mg) | 482.3 | 726.1 | 702.63 | 7560 |
| Filtrate V_{H} yield (%) | 78.8 | 82.3 | 79.5 | 78.6 |
| Filtrate V_{L} yield (%) | 79.8 | 79.6 | 78.2 | 63.9 |
| Refold Yield (μg/mL) | 38.1 | 37.8 | 68.1 | 50.1 |

Tables 6 and 7 below outline suitable operating parameters for the two depth filters utilized in clarification of anti-CD22 antibody fragments.

TABLE 6
Clarification by depth filtration

| | Target Value | Acceptable Range | Comments |
|---|---|---|---|
| C0HC Filter Flush (L/m²) | NA | ≧100 | |
| C0HC Filter EQ (L/POD) | 0.11 m² = 1.5 | | |
| | 0.55 m² = 5.3 | | |
| | 1.1 m² = 10.3 | | |
| C0HC Operating Pressure | NA | ≦20 | |
| (Δpsig) | | | |
| C0HC Capacity (L/m²) | NA | ≦61 | Includes safety factor |
| | | | of 1.5 x max filter |
| | | | capacity |
| C0HC Flux (LMH) | 100 | ≦155 | Filter flux can be |
| | | | reduced to maintain |
| | | | operating pressure |
| X0HC Filter Flush (L/m²) | NA | ≧100 | |
| X0HC Filter EQ (L/POD) | 0.11 m² = 1.5 | | |
| | 0.55 m² = 5.3 | | |
| | 1.1 m² = 10.3 | | |
| X0HC Operating Pressure | | ≦20 | |
| (Δpsig) | | | |
| X0HC Capacity (L/m²) | | ≦130 | Includes safety factor |
| | | | of 1.5 x max filter |
| | | | capacity |
| X0HC Flux (LMH) | | ≦600 | Fixed to C0HC filter |
| | | | flux, range value is |
| | | | manufacturers |
| | | | maximum |
| | | | recommended flux |
| Product Collection | | | Collect all filtrate |
| | | | post equilibration |
| Post filtration flush | 1 system hold- | | Chase product with 1 |
| | up volume | | system hold-up |
| | | | volume. End product |
| | | | collection after 1 |
| | | | system hold-up |
| | | | volume has passed |

TABLE 7

| | Trial Runs | | | | | | | | | |
|---|---|---|---|---|---|---|---|---|---|---|
| Parameter | 1 | 2 | 3 | 4 | 5 | 6 | 7 | 8 | 9 | 10 |
| COHC surface area (m²): | 7.7 | 7.7 | 7.7 | 7.7 | 7.7 | 3.52 | 3.52 | 3.52 | 3.52 | 3.52 |
| XOHC surface area (m²): | 5.5 | 5.5 | 5.5 | 5.5 | 5.5 | 2.53 | 2.53 | 2.53 | 2.53 | 2.53 |
| total surface area (m²): | 13.2 | 13.2 | 13.2 | 13.2 | 13.2 | 13.2 | 6.05 | 6.05 | 6.05 | 6.05 |
| COHC load (L/m²): | 30 | 51 | 54 | 55 | 57 | 39 | 58 | 58 | 58 | 58 |
| XOHC load (L/m²): | 41 | 71 | 75 | 77 | 80 | 55 | 80 | 818 | 81 | 81 |
| Total load (L/m²): | 17 | 30 | 31 | 32 | 33 | 23 | 33 | 34 | 34 | 34 |
| Flowrate (L/min.): | 12.1 | 8.0 | 7.9 | 16.0 | 6.5 | 9.4 | 17.69 | 15.20 | 19.98 | 15.2 |
| COHC flux (LMH): | 94 | 62 | 62 | 125 | 51 | 160 | 302 | 259 | 641 | 259 |
| XOHC flux (LMH): | 132 | 87 | 86 | 175 | 81 | 223 | 420 | 360 | 474 | 360 |
| Total filter flux (LMH): | 55 | 36 | 36 | 73 | 30 | 93 | 175 | 151 | 198 | 151 |

### Refold

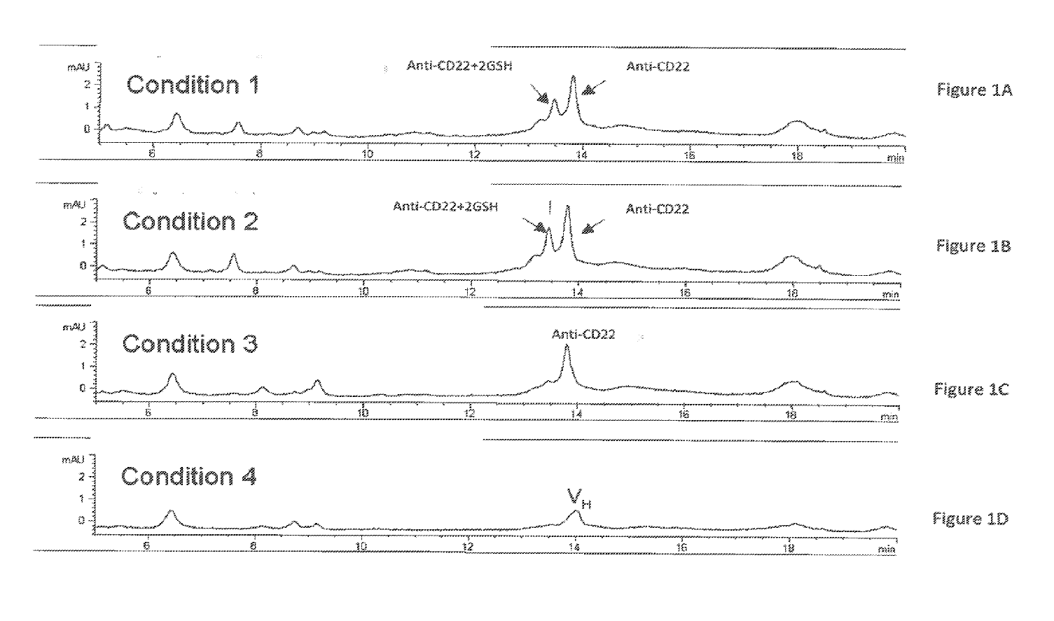

As a comparative example, a refold was carried out by concentrating clarified anti-CD22 antibody fragment material to 5mg/mL total protein by tangential flow filtration with a 5 KDa membrane. The concentrated material was 10-fold diluted into 50 mM ethanolamine, 1.0M arginine, 2 mM EDTA, 9.1 mM GSSG, and 0.91 mM glutathione (GSH), pH 9.5. The reaction was allowed to proceed at 2-8° C. for 72 hours with gentle mixing. As shown in the FIG. 1A (Condition 1), analysis of refolded samples by reverse-phase high performance liquid chromatography mass spectroscopy (RP-HPLC-MS) showed that the reaction consistently produced material containing 30-40% glutathione adduct.

In comparison, the following method was utilized to reduce or prevent the formation of glutathione adduct. An experiment was conducted to determine if changing the levels of GSH and GSSG would reduce the level of the glutathione adduct while maintaining the refold efficiency. Table 8 shows the renaturation conditions that were tested at the 0.25 kg refold scale. The GSH and GSSG were added to 50 mM ethanolamine, 1.0 mM Arginine, and 2 mM EDTA, pH 9.5 just prior to use. Condition 1 represents the comparative example discussed above. The other conditions were selected to determine if GSH was required for a successful refold and to find the minimum optimum level of GSSG.

TABLE 8

| Condition | GSH (mM) | GSSG (mM) |
|---|---|---|
| 1 | 0.91 | 9.1 |
| 2 | 0 | 9.1 |
| 3 | 0 | 0.91 |
| 4 | 0 | 0 |

The RP-HPLC-MS data showed that as expected, the glutathione adduct was present in the Condition 1 control product (see FIG. 1A). Anti-CD22 antibody containing a glutathione adduct was also produced by Condition 2 (see FIG. 1B). Both condition 1 and 2 generated material of the same concentration and proportion of glutathione adduct. The results of Condition 3 in FIG. 1C show that an anti-CD22 antibody refold product having the same concentrations as conditions 1 and 2 was formed, but that glutathione adduct containing anti-CD22 was not detected. Anti-CD22 was not produced in condition 4 (see FIG. 1D). These results indicate that GSSG is required, but GSH is not required for refolding of anti-CD22. In addition, the optimum level of glutathione disulfide (GSSG) in the reaction can be reduced by 10-fold, resulting in reduced glutathione adduct containing anti-CD22 antibody, while not impacting the yield of the antibody fragment.

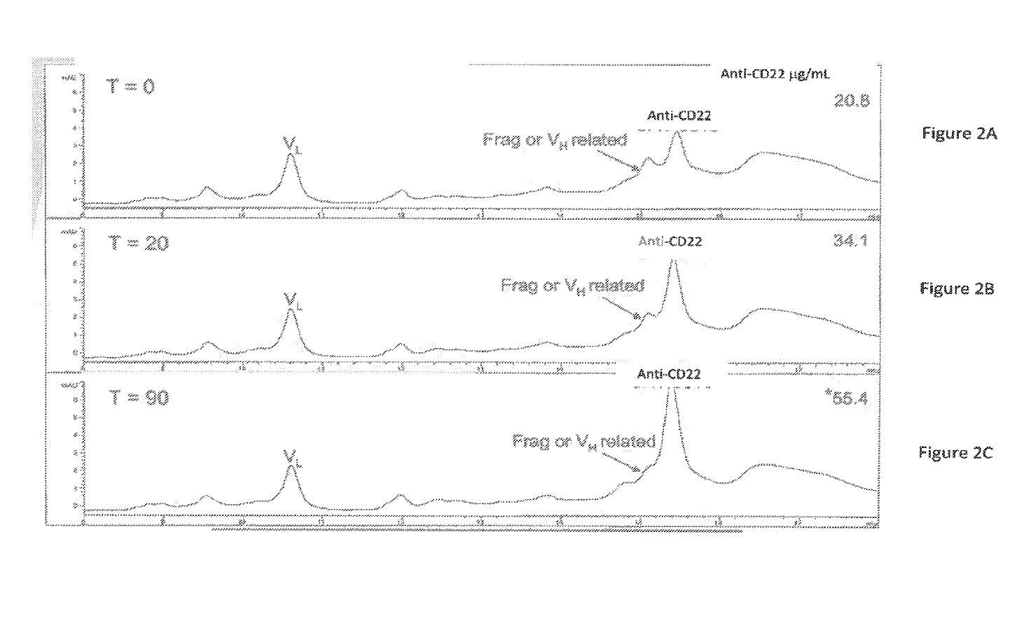

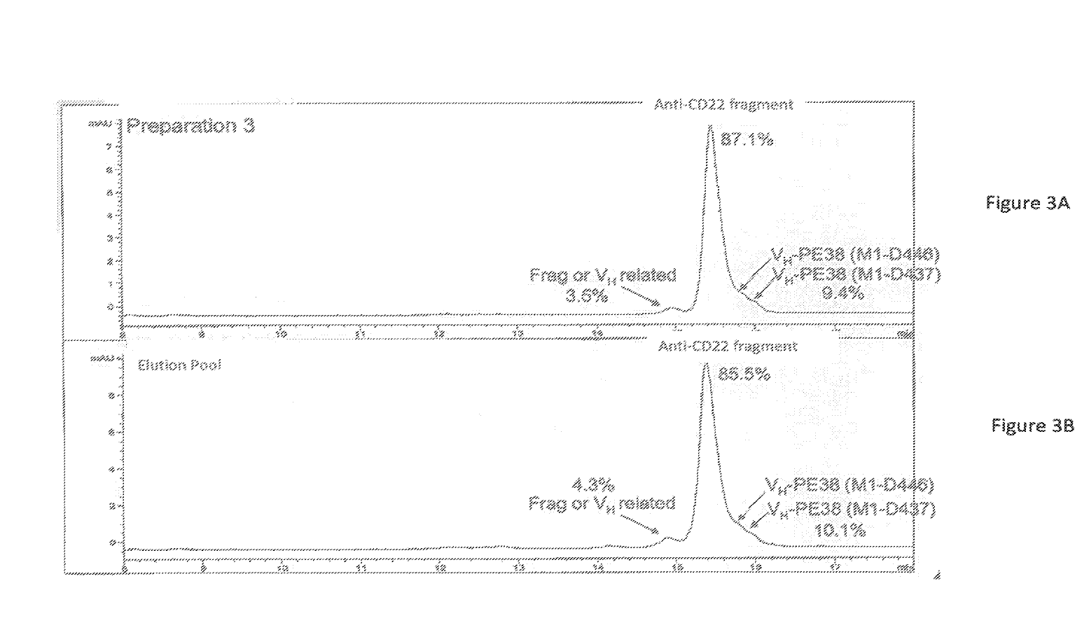

Additional results (RF-HPLC-MS) showing the refolding of anti-CD22 antibody fragment utilizing Condition 3 from above at three time points after the beginning of the incubation in refolding buffer are shown in FIG. 2A (time=0), FIG. 2B (time=20 hours) and FIG. 2B (time=90 hours). FIG. 3A and FIG. 3B show the refolded anti-CD22 fragment following additional purification.

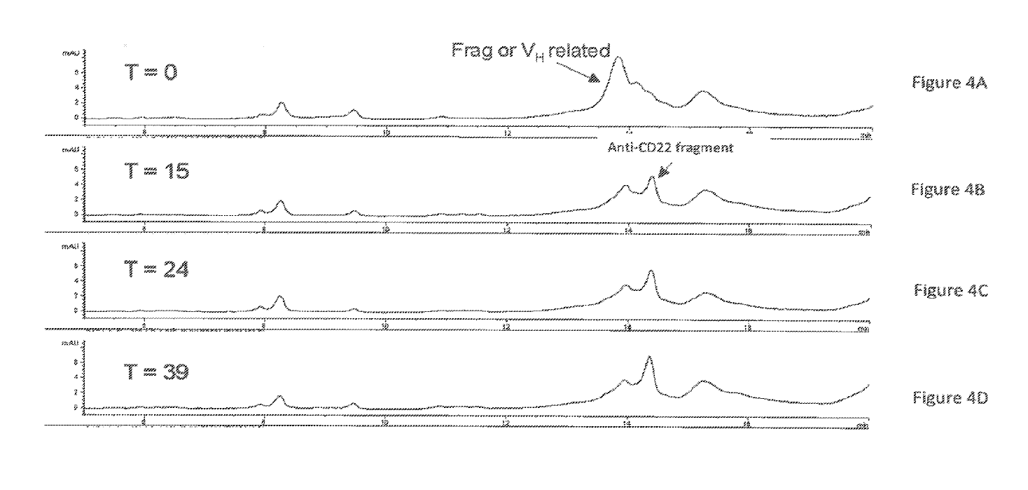

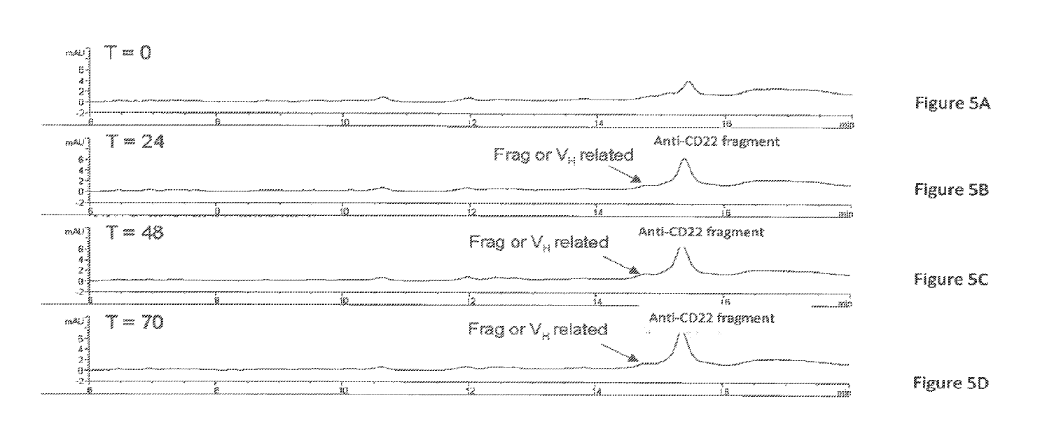

Refolding experiments using Condition 3 were performed at 5 kG and 100 kg scales with different lots of heavy and light chains to ensure that glutathione adduct containing anti-CD22 would not be formed at different scales or with different lots of inclusion body mixtures. FIGS. 4A-4D and 5A-5D show RP-HPLC-MS analyses of the 5 kg and 100 kg refold reactions, respectively, across different time points. Data for time=0, 15 hours, 24 hours and 39 hours is shown in FIGS. 4A-4D for the 5 kg refold. Data for time 0, 24 hours, 48 hours and 70 hours is shown in FIGS. 5A-5D for the 100 kg refold. The data shows that the glutathione adduct was not detected in the product and that high concentrations of anti-CD22 were formed in the scaled-up reactions (54.8 μg/mL in the 100 kg refold). The data shows that the elimination of GSH and the 10-fold decrease of GSSG resulted in the formation of anti-CD22 lacking a glutathione adduct. Refolds using this condition produced material of comparable concentration regardless of scale or lot of inclusion body.

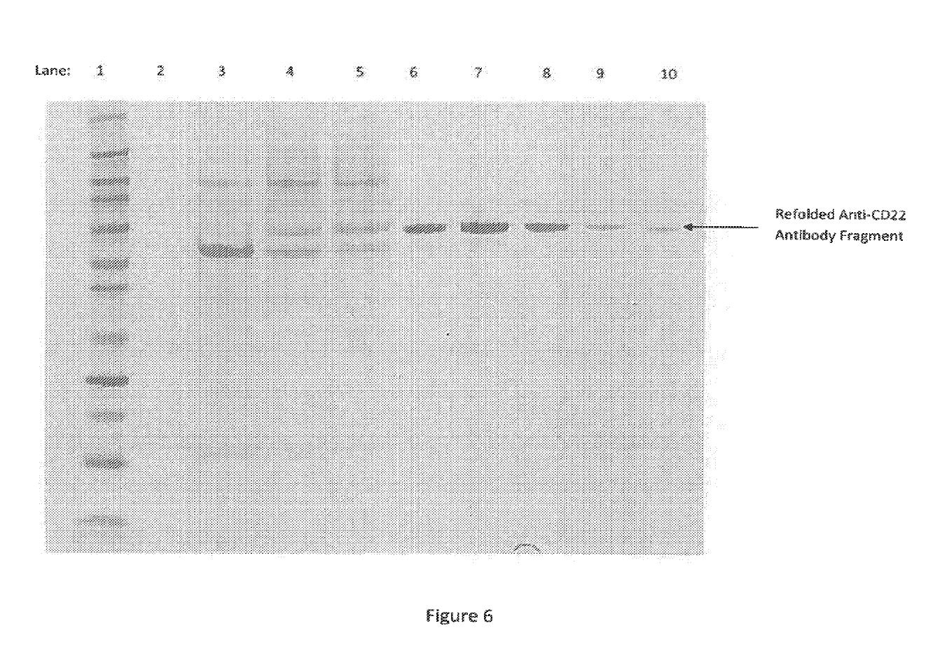

FIG. 6 shows a Western blot demonstrating the presence of anti-CD22 antibody fragment in a 100 L scale reaction. The refold was carried out for 72 hours and yielded 2.3 grams of anti-CD22 antibody fragment based on SDS-PAGE analysis. This translates to a potential yield of about 23 grams for a 1000 L reaction. Table 7 indicates the lane contents shown in the Western blot.

TABLE 7

| Lane | Content description |
|---|---|
| 1 | Molecular weight marker |
| 2 | Blank |
| 3 | Comparative refolding buffer, time 0 |
| 4 | Comparative refolding buffer, time 24 hours |
| 5 | Comparative refolding buffer, time 48 hours |
| 6 | Exemplary Refolding buffer, 1 μg of material |
| 7 | (mis-load) |
| 8 | Suitable Refolding buffer disclosed herein, 0.5 μg of |
| | material |
| 9 | Suitable Refolding buffer disclosed herein, 0.25 μg of |
| | material |
| 10 | Suitable Refolding buffer disclosed herein, 0.125 μg of |
| | material |

It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

1. A method for purifying a recombinant protein from a mixture comprising the recombinant protein and inclusion bodies, the method comprising:
a) solubilizing the mixture comprising the recombinant protein and inclusion bodies with a solubilization buffer;
b) clarifying the recombinant protein from the solubilized mixture with one or more depth filters; and
c) recovering the clarified recombinant protein,
wherein the method does not include centrifuging the solubilized mixture of recombinant protein and inclusion bodies prior to the clarifying.

2. The method of claim 1, wherein the recombinant protein comprises an antibody or an antibody fragment.

3. The method of claim 2, wherein the antibody fragment is a heavy chain (V_{H}) antibody fragment.

4. The method of claim 2, wherein the antibody fragment is a light chain (V_{L}) antibody fragment.

5. The method of claim 2, wherein the recombinant protein comprises both a V_{H} antibody fragment and a V_{L} antibody fragment.

6. The method of claim 3, wherein the V_{H} antibody fragment is an anti-CD22 V_{H} antibody fragment.

7. The method of claim 4, wherein the V_{L} antibody fragment is an anti-CD22 V_{L} antibody fragment.

8. The method of claim 5, wherein the V_{H} antibody fragment and the V_{L} antibody fragment are present at an initial molar ratio of about 1:1.

9. The method of claim 1, wherein the solubilization buffer comprises ethanolamine, arginine, EDTA, urea and DTE.

10. The method of claim 9, wherein the solubilization buffer comprises about 20 mM to about 70 mM ethanolamine, about 200 mM to about 2 M arginine, about 1 mM to about 3mM EDTA, about 5 M to about 10 M urea and about 5 mM to about 20 mM DTE, and wherein the solubilization buffer has a pH of about 10 to about 11.

11. The method of claim 1, wherein the recombinant protein is clarified with two or more depth filters.

12. The method of claim 11, wherein a first depth filter comprises cellulose fiber and diatomaceous earth, and wherein the first depth filter has a nominal micron rating of about 0.1 μm to about 1 μm.

13. The method of claim 12, further comprising clarifying with a second depth filter, wherein the second depth filter comprises cellulose fiber and diatomaceous earth, and wherein the second depth filter has a nominal micron rating of less than about 0.1 μm.

14. The method of claim 13, wherein the first depth filter is a C0HC depth filter and the second depth filter is a X0HC depth filter.

15. The method of claim 1, wherein the clarified recombinant protein yield is greater than about 70%.

16. The method of claim 1, further comprising concentrating the clarified recombinant protein and refolding the clarified recombinant protein in a protein refolding buffer.

17. The method of claim 16, wherein the refolding comprises:
a) diluting the concentrated clarified recombinant protein in the protein refolding buffer comprising about 20 mM to about 70 mM ethanolamine; about 0.5 M to about 2 M arginine; about 0.5 mM to about 3 mM EDTA; and about 0.5 mM to about 1.5 mM GSSG;
b) incubating the diluted clarified recombinant protein at a pH of about 9 to about 10 and at a temperature of about 10° C. to about 15° C., (2-15° C.) for about 48 hours to about 96 hours; and
c) recovering the recombinant protein.

18. A protein refolding buffer for refolding a solubilized recombinant protein comprising:
about 20 mM to about 70 mM ethanolamine; about 500 mM to about 2 M arginine; about 0.5 mM to about 3 mM EDTA; and about 0.5 mM to about 1.5 mM GSSG,
wherein the refolding buffer has a pH of about 9 to about 10.

19. The protein refolding buffer of claim 18, comprising:
about 50 mM ethanolamine; about 1 M arginine; about 2 mM EDTA; and
about 0.9 mM GSSG,
wherein the refolding buffer has a pH of about 9.5.

20. A method of refolding a solubilized recombinant protein, comprising:
a) concentrating the solubilized recombinant protein;
b) diluting the concentrated recombinant protein in a refolding buffer comprising:
about 20 mM to about 70 mM ethanolamine; about 500 mM to about 2 M arginine; about 0.5 mM to about 3 mM EDTA; and about 0.5 mM to about 1.5 mM GSSG;
c) incubating the diluted recombinant protein at a pH of about 9 to about 10 and at a temperature of about 10° C. to about 15° C., (2-15° C.)for about 48 hours to about 96 hours; and
d) recovering the refolded recombinant protein.

21. (canceled)

22. (canceled)

23. (canceled)

24. (canceled)

25. A method of producing a recombinant antibody fragment comprising a V_{H} antibody fragment and a V_{L} antibody fragment, the method comprising:
a) expressing a polynucleotide encoding a V_{H} antibody fragment in a first bacterial cell;
b) expressing a polynucleotide encoding a V_{L} antibody fragment in a second bacterial cell;
c) mixing the V_{H} antibody fragment and the V_{L} antibody fragment to generate a mixture, wherein the mixture further comprises inclusion bodies;
d) solubilizing the mixture comprising the V_{H} antibody fragment, the V_{L} antibody fragment and the inclusion bodies with a solubilization buffer;
e) clarifying the V_{H} antibody fragment and the V_{L} antibody fragment from the solubilized mixture with one or more depth filters;
f) recovering the clarified V_{H} antibody fragment and the clarified V_{L} antibody fragment,
wherein the method does not include centrifuging the solubilized mixture of V_{H} antibody fragment, V_{L} antibody fragment and inclusion bodies;
g) concentrating the clarified V_{H} antibody fragment and the clarified V_{L} antibody fragment;
h) diluting the concentrated clarified V_{H} antibody fragment and the concentrated clarified V_{L} antibody fragment with a refolding buffer comprising: about 20 mM to about 70 mM ethanolamine; about 0.5 M to about 2 M arginine; about 0.5 mM to about 3 mM EDTA; and about 0.5 mM to about 1.5 mM GSSG;
i) incubating the diluted clarified V_{H} antibody fragment and the diluted clarified V_{L} antibody fragment at a pH of about 9 to about 10 and at a temperature of about 10° C. to about 15° C., (2-15° C.)for about 48 hours to about 96 hours; and
j) recovering the recombinant antibody fragment.

26. (canceled)

27. (canceled)

28. (canceled)

29. (canceled)

30. (canceled)

31. (canceled)

32. (canceled)
